(19) United States
(12) Patent Application Publication
(10) Pub. No.: US 2018/0194796 A1
(43) Pub. Date: Jul. 12, 2018

(54) TRACEABLE NUCLEIC ACID MARKED FERTILIZER

(71) Applicant: APDN (B.V.I.) INC., Tortola (VG)

(72) Inventors: James A. Hayward, Stony Brook, NY (US); Michael E. Hogan, Stony Brook, NY (US); Michael Molino, Bethpage, NY (US)

(21) Appl. No.: 15/870,035

(22) Filed: Jan. 12, 2018

Related U.S. Application Data
(60) Provisional application No. 62/445,666, filed on Jan. 12, 2017.

(51) Int. Cl. C07H 21/04; C05C 1/00; C07C 211/03; C07C 229/16; C12Q 1/686; C01G 49/00

(52) U.S. Cl. CPC C07H 21/04; C05C 1/00; C01G 49/00; C07C 229/16; C12Q 1/686; C07C 211/03

(57) ABSTRACT

The invention pertains to a traceable fertilizer product marked with a nucleic acid marker. The invention further provides for a method of marking a fertilizer product with a nucleic acid marker. In addition, the invention teaches a method of using a nucleic acid marked fertilizer product to identify and validate the origin or authenticity of said fertilizer product and to perform quantitative analysis on the fertilizer product to determine if said fertilizer product has been adulterated, diluted and/or counterfeited.

### CROSS-REFERENCE TO RELATED APPLICATION

This application claims the benefit of U.S. Provisional Patent Application No. 62/445,666 filed on Jan. 12, 2017, which is hereby incorporated by reference in its entirety.

### TECHNICAL FIELD

The invention pertains to a traceable fertilizer product marked with a nucleic acid marker. The invention further provides for a method of marking a fertilizer product with a nucleic acid marker. In addition, the invention teaches a method of using a nucleic acid marked fertilizer product to identify and validate the origin or authenticity of said fertilizer product and to perform quantitative analysis on the fertilizer product to determine if said fertilizer product has been adulterated, diluted and/or counterfeited.

### BACKGROUND

A fertilizer product is any material of natural or synthetic origin that is applied to soils or to plant tissues that supplies one or more essential plant nutrients necessary for plant growth. Fertilizers generally fall into several classes. These classes include nitrogen fertilizers, phosphate fertilizers, potassium fertilizers (the foregoing called “straight fertilizers), compound fertilizers (multi-nutrient fertilizers) and organic fertilizers. Straight fertilizers contain a single plant nutrient. Multi-nutrient fertilizers are comprised of two or more plant nutrient compounds.

Nitrogen is an essential plant nutrient that facilitates leaf growth. Potassium is an essential plant nutrient that facilitates the growth of plant stems, flowers and fruits, and also facilitates the movement of water within a plant. Phosphorus promotes stem growth, flowers, seeds and fruit bearing. Calcium, magnesium, sulfur, copper, iron and other compounds are also essential plant nutrients.

Nitrogen-based fertilizers are widely used in agriculture. One of the most common nitrogen-based straight fertilizers is ammonium nitrate (NH₄NO₃). Ammonium nitrate is commonly used world-wide due to its low cost and high nitrogen content (about 34%). In addition, in normal applications ammonium nitrate is more stable and less prone to atmospheric nitrogen loss than other nitrogen-based straight fertilizers such as urea.

In addition to being a common nitrogen-based straight fertilizer, ammonium nitrate can also be used to form various explosive mixtures. When ammonium nitrate is combined with a primary explosive, such as aluminum powder or fuel oil, a powerful explosive mixture can be created. For example, ANFO, a powerful and low-cost industrial explosive is comprised of 94% ammonium nitrate and 6% fuel oil. Ammonium nitrate is often used by malicious actors to create explosive mixtures or improvised explosive devices (“IEDs”). Examples of ammonium nitrate based explosive devices are the Oklahoma City bombing that occurred in 1995 and the 2011 bombing in Oslo, Norway. In addition, many IEDs use ammonium nitrate based explosives.

Due to ammonium nitrate's potential to be used as explosive material, several countries have created regulatory measures around its sale. Currently the United States Department of Homeland Security (DHS) has proposed the Ammonium Nitrate Security Program that would track the sale of ammonium nitrate in the United States through user and purchaser registration and vetting.

Fertilizer is a valuable commodity across the world. It is often counterfeited and diluted by the time it reaches the end consumer. Counterfeited and/or diluted fertilizer has much lower efficacy and can result in lower crop yield or total crop loss.

There is a need to ensure the quality and authenticity of fertilizers to prevent counterfeit, sub-standard, and adulterated fertilizers from impacting crop yields, profitability, and causing significant environmental damage. There is also a need to be able to trace the origin of fertilizers when used for illegal purposes.

### SUMMARY

The inventors have discovered a novel means of tagging fertilizer with a nucleic acid marker in an efficient manner that protects the nucleic acid marker from degradation from the chemicals present in fertilizer. The nucleic acid marker may be easily recovered and separated from the fertilizer and then analyzed through standard methodologies. The ability for the nucleic acid marker to be separated from the fertilizer is necessary for authentication purposes because fertilizer inhibits PCR based analysis.

The inventors have also found a means of attaching a nucleic acid marker to a ferrous compound and incorporating the nucleic acid tagged ferrous compounds into fertilizer. The ferrous compound may also incorporate a flourophore or other optical marker. The magnetic nature of the ferrous compound assists in recover of the nucleic acid taggant for subsequent isolation, amplification, and authentication.

In one embodiment, the present invention provides for a traceable nucleic acid marked fertilizer product. The nucleic acid marked fertilizer product includes either a straight fertilizer and/or multi-nutrient fertilizer and a nucleic acid marker. The nucleic acid marker may be a DNA or RNA marker of a known nucleotide sequence. The nucleic acid marker may be directly incorporated in the fertilizer product or it may be incorporated with the aid of a solvent or affixed to a carrier. The DNA marker may also include a fluorophore or other optical reporter and may be alkaline activated.

In another embodiment, the present invention provides for a method of marking a fertilizer product to identify and validate the origin and/or authenticity of said fertilizer product in the stream of commerce. This method includes adding a nucleic acid marker to straight fertilizer or multi-nutrient fertilizer during a step in the production process of said fertilizer product, thereby incorporating the nucleic acid marker into the fertilizer product; introducing the nucleic acid marked fertilizer product into the stream of commerce; sampling a fertilizer product from the stream of commerce; detecting the presence or absence of a nucleic acid marker in the sampled fertilizer product; and thereby identifying and/or verifying the origin and authenticity of the fertilizer product.

In another embodiment, the present invention provides for a method of marking an ammonium nitrate fertilizer product with a nucleic acid marker to identify the origin of same. This methods includes the steps of: adding a nucleic acid marker to an ammonium nitrate fertilizer product during a step in the production process of the fertilizer product, thereby created a nucleic acid marked ammonium nitrate fertilizer product; introducing the nucleic acid marked ammonium nitrate fertilizer product into the stream of commerce; collecting a quantity of ammonium nitrate fertilizer product after an explosive detonation; sampling the collected ammonium nitrate fertilizer product; detecting the presence or absence of a nucleic acid marker in collected the ammonium nitrate fertilizer product; and identifying the origin of the ammonium nitrate fertilizer product based upon the nucleic acid marker detected.

One aspect of the invention relates to a method of making a nucleic acid tagged fertilizer, the method including contacting a nucleic acid taggant with a fertilizer coating to produce a nucleic acid tagged fertilizer coating; and adding the nucleic acid tagged fertilizer coating to a fertilizer to form a nucleic acid tagged fertilizer, wherein the fertilizer is pelletized. The fertilizer coating is preferably an anti-caking agent. Preferred anti-caking agents include clay, talc, chalk, petroleum oils, waxes, polyalcohol, fatty amines, cationic surfactants based on fatty amines, alkyl-aryl sulphonates, mineral oils, alkyl phosphoric esters, paraffins, paraffin oil, and combinations thereof. The most preferred anti-caking agent includes a fatty amine.

The nucleic acid tagged fertilizer coating is preferably added to the fertilizer by tumble coating to ensure adequate coating of the fertilizer. The nucleic acid taggant is preferably present at the level of about 0.1 nanograms to 25 micrograms per kilogram of fertilizer coating. More preferably, the nucleic acid taggant is present at the level of about 5 micrograms per ton of fertilizer.

Another aspect of the invention relates to a method of making a nucleic acid tagged fertilizer including contacting a nucleic acid taggant, one or more antioxidants, and a wetting agent with a ferrous compound to produce a nucleic acid tagged ferrous compound; and adding the nucleic acid tagged ferrous compound to a fertilizer coating to form a nucleic acid tagged fertilizer. The ferrous compound may include iron, nickel, cobalt, or alloys thereof. Preferably the ferrous compound is ferrite. The ferrite compound may be coated with a UV fluorescent dye.

The nucleic acid tagged ferrous compound may be present in about 0.1 μg to about 10 μg per ton of fertilizer.

Preferably, the one or more antioxidants are selected from the group consisting of sodium ferulate, EDTA, EDTA tetrasodium dehydrate, and combinations thereof. The wetting agent is preferably selected from the group consisting of octylphenoxypolyethoxyethanol, sorbitan monolaurate, sorbitan monooleate, polyethylene glycol sorbitan monolaurate, sorbitane trioleate, polyethylene glycol 400, and combinations thereof. Most preferably, the ratio of wetting agent to ferrous compound is about 2:1.

The fertilizer may be powdered or pelletized. When the fertilizer is pelletized, the nucleic acid tagged ferrous compound may be combined with a fertilizer coating before being added to the pelletized fertilizer.

The fertilizer coating is preferably an anti-caking agent selected from the group consisting of clay, talc, chalk, petroleum oils, waxes, polyalcohols, fatty amines, cationic surfactants based on fatty amines, alkyl-aryl sulphonates, mineral oils, alkyl phosphoric esters, paraffins, paraffin oil, and combinations thereof.

When the fertilizer is pelletized, the mixture of the nucleic acid tagged ferrous compound and fertilizer coating may be added to the fertilizer by tumble coating.

The nucleic acid taggant is most preferably a DNA taggant. The ferrous compound preferably has a particle size of about 1 nanometer to about 500 microns.

Another aspect of the invention relates to a fertilizer composition including fertilizer and a nucleic acid tagged ferrite wherein the ferrite is present in about 0.1 μg to about 10 μg per ton of fertilizer. Preferably, the nucleic acid tagged ferrite is DNA tagged ferrite. Then the fertilizer is pelletized, it may be coated with an anti-caking agent containing the nucleic acid tagged ferrite.

### BRIEF DESCRIPTION OF THE FIGURES

FIG. 1 shows a process for using a nucleic acid marked fertilizer product to identify and validate the origin or authenticity of said fertilizer product

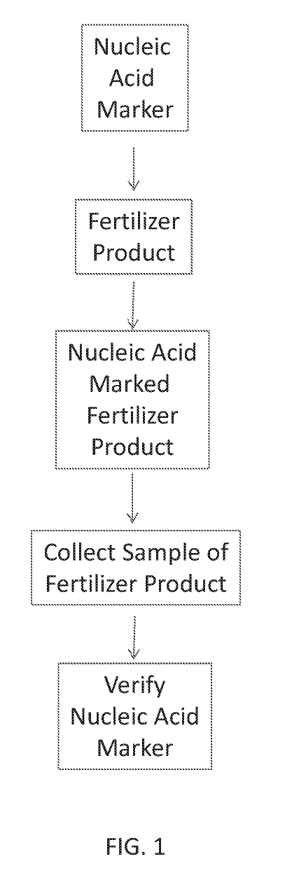

FIG. 2 show the process of using a nucleic acid marked ammonium nitrate fertilizer product to identify the origin said fertilizer product

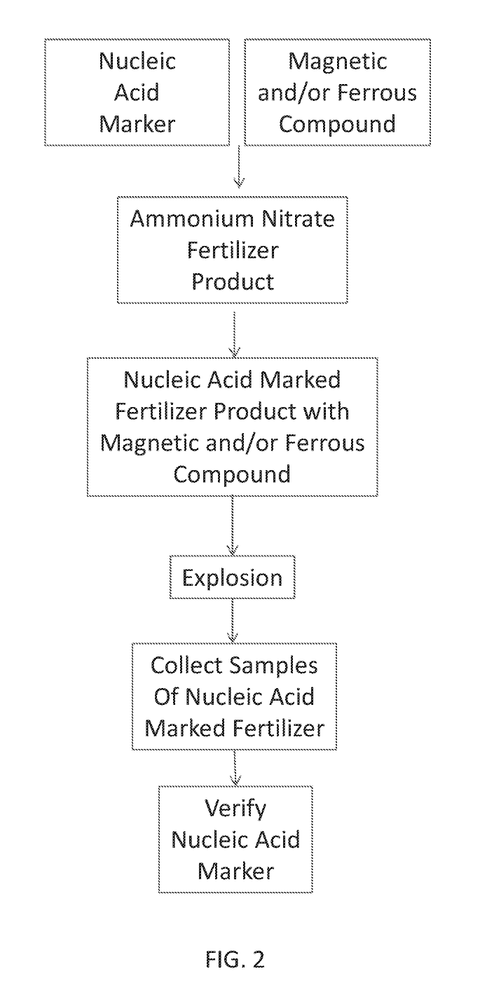

### DETAIL DESCRIPTION

Nucleic Acid Tagged Fertilizer Product

A traceable nucleic acid marked fertilizer product is disclosed herein. In one embodiment, the nucleic acid marked fertilizer product is comprised of a fertilizer product and nucleic acid marker. The fertilizer product may be straight fertilizer and/or multi-nutrient fertilizer, including but not limited to straight ammonium nitrate fertilizer. The nucleic acid marker may be a detectable DNA or RNA marker of a known nucleotide sequence. The DNA or RNA marker may be single or double stranded DNA or RNA. The nucleic acid marker may be incorporated within a fertilizer product and/or fertilizer pellet or may be applied to the exterior surface of the fertilizer product only.

The fertilizer product may be pelletized or non-pelletized. Non-pelletized fertilizer includes fertilizer in powder form. The nucleic acid marker may be comprised of DNA (deoxyribonucleic acid) or RNA (ribonucleic acid) extracted from biological material and may be from about 20 bases to about 10,000 bases in single strand length, or about 20 base pairs to about 10,000 base pairs in double strand length. In one embodiment, the DNA may be a novel DNA sequence formed of DNA that is entirety derived from the DNA of the biological material being fertilized. For example, the nucleic acid marker may be formed from a novel sequence of DNA derived exclusively from a specific plant species. Thus, if for example a nucleic acid marker fertilizer product was to be used exclusively on corn, the nucleic acid marker may be derived exclusively from corn based DNA. The nucleic acid marker may also be comprised of one or more synthetic DNA oligonucleotides constructed via DNA synthesis techniques.

Additionally, the specific sequence of DNA or RNA may act in the same manner as a bar code to provide information regarding origin of the fertilizer, date of production, lot number, composition, manufacturer etc. The specific quantity of DNA recovered per sample could also provide information whether a specific batch of fertilizer was diluted by counterfeiters.

In one embodiment, the nucleic acid marker may be alkaline activated as described in US patent application publication US 2014-0256881 A1 “Alkaline Activation For Immobilization of DNA Taggants” of Berrada et al. the entire disclosure of which is hereby incorporated by reference. In one embodiment, NaOH may be used for alkaline activation. NaOH may also be used for normalizing PH within the fertilizer product. In another embodiment, the nucleic acid marker may be treated with an additive, wherein the additive is a polyol, diol, glycol, starch, or pyrrolidone. Another embodiment of the invention includes a nucleic acid marker suspended in a solvent carrier.

In another embodiment the nucleic acid marked fertilizer may also include a quantity of ferrous metals or other magnetic compounds. The ferrous metals or other magnetic compounds may be in a powdered form. The ferrous metals or other magnetic compounds may be of submicron particle size. In one embodiment, submicron particles of ferrous metals are coated with a nucleic acid marker and incorporated into the fertilizer product.

In another embodiment, a fertilizer composition is provided that includes fertilizer and a nucleic acid tagged ferrite, wherein the ferrite is present in about 0.1 μg to about 10 μg per ton of fertilizer. Preferably, the nucleic acid tagged ferrite is a DNA tagged ferrite particle ranging in size from 1 nanometer to 500 microns. The DNA tagged ferrite may include alkaline activated DNA. If the fertilizer is pelletized, then the fertilizer may be coated with an anti-caking agent containing the nucleic acid tagged ferrite.

Method of Marking a Fertilizer Product to Identify and/or Validate the Origin or Authenticity of Said Fertilizer Product

In an embodiment, the present invention provides for a method of marking a fertilizer product with a nucleic acid marker to identify and/or validate the origin or authenticity of same. The fertilizer product may be pelletized or non-pelletized. This method includes the steps of: adding a nucleic acid marker to a fertilizer product during a step in the production process of the fertilizer product, thereby creating a nucleic acid marked fertilizer product; introducing the nucleic acid marked fertilizer product into the stream of commerce; collecting a quantity of fertilizer product from the stream of commerce; sampling the collected fertilizer product; detecting the presence or absence of a nucleic acid marker in a fertilizer product; and identifying and/or verifying the origin and/or authenticity of the fertilizer product based upon the presence or absence of a nucleic acid marker in the collected fertilizer product.

The nucleic acid marker may be added to a fertilizer product during any step in the production process. The added nucleic acid marker may be alkaline activated as described in US patent application publication US 2014-0256881 A1 “Alkaline Activation For Immobilization of DNA Taggants” of Berrada et al. the entire disclosure of which is hereby incorporated by reference. In one embodiment, NaOH may be used for alkaline activation. NaOH may also be used for normalizing PH. In another embodiment, the nucleic acid marker may be treated with an additive, wherein the additive is a polyol, diol, glycol, starch, or pyrrolidone.

In one embodiment, the nucleic acid marker is added to the fertilizer product's master batch. In another embodiment, the nucleic acid marker is added to a single ingredient of the fertilizer product's formulation, such as the fertilizer's colorant. In one embodiment the nucleic acid marker is sprayed in solution onto a finished fertilizer product. The nucleic acid marker may also be incorporated into any exterior coating of a fertilizer product.

The detection of the presence or the absence of the nucleic acid marker in a fertilizer product may be undertaken by any DNA testing or analysis methodologies known in the art.

A quantity of fertilizer product may be obtained from any fertilizer of interest. Fertilizer that is comprised of, or sourced from, a nucleic acid marked fertilizer product is preferred. A quantity of fertilizer product may also be obtained from the soil upon which the fertilizer product was applied or from the external surface of the plants upon which fertilizer product has been applied.

In another embodiment, the present invention provides for a method of marking an ammonium nitrate fertilizer product with a nucleic acid marker to identify the origin of same.

This methods includes the steps of: adding a nucleic acid marker to a ammonium nitrate fertilizer product during a step in the production process of the fertilizer product, thereby created a nucleic acid marked ammonium nitrate fertilizer product; introducing the nucleic acid marked ammonium nitrate fertilizer product into the stream of commerce; collecting a quantity of ammonium nitrate fertilizer product after an explosive detonation; sampling the collected ammonium nitrate fertilizer product; detecting the presence or absence of a nucleic acid marker in the collected ammonium nitrate fertilizer product; and identifying the origin of the ammonium nitrate fertilizer product based upon a nucleic acid marker detected.

In a preferred embodiment, the method of making a nucleic acid tagged fertilizer, includes contacting a nucleic acid taggant with a fertilizer coating to produce a nucleic acid tagged fertilizer coating; and adding the nucleic acid tagged fertilizer coating to a fertilizer to form a nucleic acid tagged fertilizer. The nucleic acid taggant is contacted with a fertilizer coating by methods known in the art such as mixing or stirring.

The fertilizer coating is preferably a fertilizer anti-caking agent. Fertilizer anti-caking agents include all agents which prevent dry fertilizer from clumping during storage and transport. Caking is usually caused by the crystallization of water soluble salts and the resulting bridges between the surfaces of the fertilizer particles. Anti-caking agents, i.e., conditioning agents, contain surface active agents to disrupt typical fertilizer caking mechanisms. These agents are usually formulated with other components to reduce dust formation which can lead to caking by promoting crystal bridging.

Examples of anti-caking agents include, but are not limited to, clay, talc, chalk, petroleum oils, waxes, polyalcohols, fatty amines, cationic surfactants based on fatty amines, alkyl-aryl sulphonates, mineral oils, alkyl phosphoric esters, paraffins, paraffin oil, and combinations thereof. Fatty amines include primary fatty amines, i.e., monoalkylamines in which the alkyl group is of fatty origin. A preferred anti-caking agent is Lilamin® produced by Ceca, a subsidiary of the Arkema Group, which is an oily base containing fatty amines and fatty derivatives. The anti-caking agent may also include a gum to increase viscosity. Typical gums include xantham gum, guar gum, acacia senegal gum, and combinations thereof. Solagum® produced by Seppic is a preferred gum which is made up of acacia Senegal gum and xantham gum. Solagum® may be added to Lilamin® in up to about 1%, 2%, 3%, 4%, 5%, 6%, 7%, 8%, 9%, or 10% in order to achieve a good viscosity when combined with the nucleic acid taggant.

The tagged fertilizer coating is then added to a fertilizer to form a nucleic acid tagged fertilizer. To ensure adequate coating of the fertilizer, specialized equipment is preferably used such as a coating drum with a spray bar and fins to tumble coat the fertilizer. Spraying bars projected forward from the back of the drum are used to add the coating to the fertilizer. A dye colorant may also be added to the coating drum through the spaying bars or it may be added directly into the coating mixture.

The amount of fertilizer coating may be determined by a person having skill in the art so that the fertilizer does not clump during transport and storage. The amount varies depending upon the particular fertilizer coating being used. The following percentages may be combined to create a minima, a maxima, or range for the percentage of fertilizer coating present based upon the weight of the fertilizer: 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.55%, and 0.6%. For example, in a preferred embodiment, the amount of fertilizer coating may be present in about 0.01% to about 0.2% based upon the total weight of the fertilizer. In one preferred embodiment, about 0.333 kg Lilamin® is used to mark 1 ton of fertilizer (0.0367% coating to fertilizer).

Small amounts of nucleic acid taggant are added to the fertilizer coating to create a nucleic acid tagged fertilizer coating. For example, the amount of nucleic acid taggant added to a fertilizer coating may range from micrograms (10⁻⁶ g) to less than a nanogram (10⁻⁹ g) per kilogram of fertilizer coating. In a preferred embodiment, the amount of nucleic acid taggant added to a fertilizer coating may range from 0.1 nanograms (10⁻¹⁰ g) to 25 micrograms (25×10⁻⁶ g) of nucleic acid taggant added per kilogram of fertilizer coating. In another embodiment, the amount of nucleic acid taggant is less than 1 ppt (10⁻¹²) w/w of the fertilizer coating.

Suitable exemplary ranges of nucleic acid tagging loading for fertilizer coating include for instance:

A range from about 0.1 nanogram (10⁻¹⁰ g) to about 15 microgram (15×10⁻⁶ g) of nucleic acid taggant added per kilogram (10³ g) of fertilizer coating.

A range from about 0.1 nanogram (10×10⁻¹⁰ g) to about 1 microgram (10⁻⁶ g) of nucleic acid taggant added per kilogram (10³ g) of fertilizer coating.

A range from about 0.1 nanogram (10×10⁻¹⁰ g) to about 100 nanograms (100×10⁻⁹ g) nucleic acid taggant added per kilogram (10³ g) of fertilizer coating.

A range from about 0.1 nanogram (10×10⁻¹⁰ g) to about 10 nanograms (10×10⁻⁹ g) of nucleic acid taggant added per kilogram (10³ g) of fertilizer coating.

A range from about 1 picograms (1×10⁻¹² g) to about 100 microgram (100×10⁻⁶ g) of nucleic acid taggant added per kilogram (10³ g) of fertilizer coating.

A range from about 1 femtogram (10⁻¹⁵ g) to about 1 microgram (10⁻⁶ g) of nucleic acid taggant added per kilogram (10³ g) of fertilizer coating.

A range from about 10 femtograms (10×10⁻¹⁵ g) to about 100 nanograms (100×10⁻⁹ g) of nucleic acid taggant added per kilogram (10³ g) of fertilizer coating.

A range from about 100 femtograms (100×10⁻¹⁵ g) to about 10 nanograms (10×10⁻⁹ g) of nucleic acid taggant added per kilogram (10³ g) of fertilizer coating.

A range from about 1 picograms (1×10⁻¹² g) to about 1 nanogram (1×10⁻⁹ g) of nucleic acid taggant added per kilogram (10³ g) of fertilizer coating.

The amount of nucleic acid taggant per ton of fertilizer may range from 3 micrograms to 8 micrograms. In a preferred embodiment, 5 micrograms of nucleic acid taggant per ton of fertilizer is used (5 picograms of nucleic acid taggant per gram of fertilizer). The nucleic acid tagged fertilizer coating may also include dyes as mentioned above.

The dyes may be any dyes used in the manufacture of fertilizer. The following percentages may be combined to create a minima, a maxima, or range for the percentage of dye present based upon the total weight of the nucleic acid tagged fertilizer coating: 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, and 30%. For example, dyes may be present in about 0% to about 25% based upon the total weight of the nucleic acid tagged fertilizer coating.

The nucleic acid tagged fertilizer may be authenticated by first taking a sample of the coated fertilizer by methods such as wiping the surface of the fertilizer with a cotton swab wetted with sterile water. Alternatively, the cotton swab may be wet with methyl ethyl ketone or another alternative competitive binding substance to detach the coating from the fertilizer. In the case of pelletized fertilizer, one or more fertilizer pellets may be swabbed for comparative analysis.

All or part of the cotton swab, now containing a quantity of nucleic acid tagged fertilizer coating, is placed into a 0.1 mL vessel. 20 μL of extraction buffer is added to the vessel and the sample is heated at 95° C. for 10 minutes. Once the nucleic acid taggant is removed from the fertilizer and isolated, nucleic acid amplification and/or taggant sequence detection techniques may be employed to amplify and identify the nucleic acid taggant. For example, in a PCR-based identification method, the nucleic acid, e.g., DNA taggants recovered from the fertilizer are amplified by polymerase chain reaction (PCR) and resolved by gel electrophoresis, capillary electrophoresis, or the like. Since the nucleic acid sequence of the nucleic acid taggants of the present invention are unique and specific to the tagged fertilizer, the nucleic acid taggant will be amplified during PCR only by use of primers having specific sequences complementary to a portion of the unique taggant sequence. Through this procedure, if the examined fertilizer carries the nucleic acid taggant, the PCR procedure will amplify the extracted nucleic acid to produce known and detectable amplicons of a predetermined size and a sequence. In contrast, if the sample recovered from the examined fertilizer does not include the unique nucleic acid sequence corresponding to the taggant of the authentic fertilizer, there will likely be no amplified nucleic acid product, or if the primers do amplify the recovered nucleic acid to produce one or more random amplicons, these one or more amplicons cannot have the unique taggant nucleic acid sequence and/or length from the authentic fertilizer. Furthermore, the random amplicons derived from counterfeit fertilizer are also of random lengths and the likelihood of producing amplicons of the exact lengths specified by the taggant-specific primers is very small. Therefore, by comparing the length and quantity of PCR amplicons, the authenticity of labeled objects can be verified, non-authentic fertilizer can be screened and rejected, and anti-counterfeit screening purposes are then achieved. The DNA may also be amplified by any other known amplification techniques such as loop mediated isothermal amplification, rolling circle amplification, nucleic acid sequence base amplification, ligase chain reaction or recombinase polymerase amplification (RPA).

The quantity of amplicons and the lengths of the amplicons can be determined after any molecular weight or physical dimension-based separation, such as for instance and without limitation, gel electrophoresis in any suitable matrix medium for example in agarose gels, polyacrylamide gels or mixed agarose-polyacrylamide gels, or the electrophoretic separation can be in a slab gel or by capillary electrophoresis.

In addition, any known sequence detection and/or identification technique may be used to detect the presence of the nucleic acid taggant such as, for example, hybridization with a marker-sequence specific nucleic acid probe, an in situ hybridization method (including fluorescence in situ hybridization: FISH), as well as amplification and detection via PCR, such as quantitative (qPCR)/real time PCR (RT-PCR). Isothermal amplification and taggant sequence detection may also be performed with the aid of an in-field detection device such as the T-16 Isothermal Device manufactured by TwistDX, Limited (Hertfordshire, United Kingdom). Interrogation may also be accomplished via various in-field techniques as disclosed in Jung et al. (U.S. Ser. No. 14/471,722) as well as through the use of infield microarray system utilizing a sequence specific quenched florescent probe system.

In an additional embodiment, the present invention provides for a method of marking an ammonium nitrate fertilizer product with nucleic acid marker and a quantity of magnetic and/or ferrous compounds. This methods includes the steps of: adding a nucleic acid marker and a magnetic and/or ferrous compound to the ammonium nitrate fertilizer product during a step in the production process of the fertilizer product thereby created a nucleic acid marked ammonium nitrate fertilizer product that also includes a magnetic and/or ferrous compound; introducing the nucleic acid marked ammonium nitrate fertilizer product also containing a magnetic and/or ferrous compound into the stream of commerce; collecting a quantity of nucleic acid marked ammonium nitrate fertilizer product also containing a magnetic and/or ferrous compound after an explosive detonation via the use of a magnetic field; sampling the collected ammonium nitrate fertilizer product; detecting the presence or absence of a nucleic acid marker in the collected ammonium nitrate fertilizer product; and identifying the origin of the ammonium nitrate fertilizer product based upon the nucleic acid marker detected.

The magnetic and/or ferrous compounds may be incorporated into the ammonium nitrate fertilizer product at any step in production. The ferrous metals or other magnetic compounds may be in powdered form. The ferrous metal or other magnetic compounds may be comprised of submicron sized particles. In one embodiment, submicron particles of ferrous metals may be coated with a nucleic acid marker and added to an ammonium nitrate fertilizer product.

Another embodiment relates to a method of making a nucleic acid tagged fertilizer, including contacting a nucleic acid taggant, an antioxidant, and a wetting agent with a ferrous compound to produce a nucleic acid tagged ferrous compound; and adding the nucleic acid tagged ferrous compound to a fertilizer to form a nucleic acid tagged fertilizer.

The nucleic acid taggant as described above is preferably DNA. The amount of nucleic acid taggant added to a ferrous compound may range from micrograms (10⁻⁶ g) to less than a nanogram (10⁻⁹ g) per kilogram of ferrous material. In a preferred embodiment, the amount of nucleic acid taggant added to the ferrous compound may range from 0.1 nanograms (10⁻¹⁰ g) to 15 micrograms (15×10⁻⁶ g) of nucleic acid taggant added per kilogram of ferrous material. In another embodiment, the amount of nucleic acid taggant is less than 1 ppt (10⁻¹²) w/w of the ferrous material.

Suitable exemplary ranges of nucleic acid tagging loading for ferrous compounds include for instance:

A range from about 0.1 nanogram (10⁻¹⁰ g) to about 10 microgram (10×10⁻⁶ g) of nucleic acid taggant added per kilogram (10³ g) of ferrous compound.

A range from about 0.1 nanogram (10×10⁻¹⁰ g) to about 1 microgram (10⁻⁶ g) of nucleic acid taggant added per kilogram (10³ g) of ferrous compound.

A range from about 0.1 nanogram (10×10⁻¹⁰ g) to about 100 nanograms (100×10⁻⁹ g) nucleic acid taggant added per kilogram (10³ g) of ferrous compound.

A range from about 0.1 nanogram (10×10⁻¹⁰ g) to about 10 nanograms (10×10⁻⁹ g) of nucleic acid taggant added per kilogram (10³ g) of ferrous compound.

A range from about 1 picograms (1×10⁻¹² g) to about 100 microgram (100×10⁻⁶ g) of nucleic acid taggant added per kilogram (10³ g) of ferrous compound.

A range from about 1 femtogram (10⁻¹⁵ g) to about 1 microgram (10⁻⁶ g) of nucleic acid taggant added per kilogram (10³ g) of ferrous compound.

A range from about 10 femtograms (10×10⁻¹⁵ g) to about 100 nanograms (100×10⁻⁹ g) of nucleic acid taggant added per kilogram (10³ g) of ferrous compound.

A range from about 100 femtograms (100×10⁻¹⁵ g) to about 10 nanograms (10×10⁻⁹ g) of nucleic acid taggant added per kilogram (10³ g) of ferrous compound.

A range from about 1 picograms (1×10⁻¹² g) to about 1 nanogram (1×10⁻⁹ g) of nucleic acid taggant added per kilogram (10³ g) of ferrous compound.

The DNA may also be alkaline activated.

The antioxidant is selected from the group consisting of sodium ferulate, Ethylenediaminetetraacetic acid (EDTA), ETDA tetrasodium dehydrate, other similar antioxidants and combinations thereof. In an exemplary embodiment, ETDA tetrasodium dehydrate is used at a concentration of 0.003M and sodium ferulate is used at a concentration of approximately 0.006M.

The wetting agent may be any commonly used wetting agent known in the art. For example, the wetting agent may be selected from the group consisting of, nonionic detergents such as octylphenoxy poly(ethyleneoxy)ethanol, branched (sold as IGEPAL® CA-630 by Millipore Sigma, formerly sold as Nonidet P40 by Millipore Sigma), nonionic surfactants such as sorbitan monolaurate, sorbitan monooleate, polyethylene glycol sorbitan monolaurate (sold as TWEEN® 20 by Millipore Sigma), sorbitane trioleate (sold as Span® 85 by Millipore Sigma), polyethylene glycol 400, and combinations thereof. In an exemplary embodiment, octylphenoxy poly(ethyleneoxy)ethanol, branched is used in a concentration of 0.01% of total solution volume.

Prior to application to a ferrous compound, the nucleic acid taggant, antioxidant and wetting agent are combined in an aqueous solution. A mass excess of the taggant, antioxidant and wetting agent solution is applied to the ferrous compound. For example, the ratio of the solution to ferrous compound is preferably about 4:1 to about 3:2. Most preferably, the ratio of the solution to the ferrous compound is about 2:1.

The ferrous compound may be any metal or alloy that is ferromagnetic. For example, the ferrous compound may include iron, nickel, cobalt, or alloys thereof. A preferred ferrous compound is ferrite. The ferrite may be coated with a UV fluorescent dye to assist in recovery such as commercially available MagnaGlo 14A Powder manufactured by Magnaflux. MagnaGlo 14A is a dry, free flowing, brown magnetic powder which fluoresces bright yellow-green under a black light. The ferrous compound may have a particle size ranging from about 1 nanometer to about 500 microns.

The following number of μg may be combined to create a minima, a maxima, or range for amount of nucleic acid tagged ferrous compound may be present per ton of fertilizer: 0.01 μg, 0.02 μg, 0.03 μg, 0.04 μg, 0.05 μg, 0.06 μg, 0.07 μg, 0.08 μg, 0.09 μg, 0.1 μg, 0.2 μg, 0.3 μg, 0.4 μg, 0.5 μg, 0.6 μg, 0.7 μg, 0.8 μg, 0.9 μg, 1 μg, 1.1 μg, 1.2 μg, 1.3 μg, 1.4 μg, 1.5 μg, 1.6 μg, 1.7 μg, 1.8 μg, 1.9 μg, 2 μg, 2.1 μg, 2.2 μg, 2.3 μg, 2.4 μg, 2.5 μg, 2.6 μg, 2.7 μg, 2.8 μg, 2.9 μg, 3 μg, 4 μg, 5 μg, 6 μg, 7 μg, 8 μg, 9 μg, and 10 For example, the nucleic acid tagged ferrous compound may be present in about 0.1 μg to about 10 μg per ton of fertilizer. Preferably, the nucleic acid tagged ferrous compound may be present in about 1 μg per ton of fertilizer.

The fertilizer may be powdered or pelletized. If the fertilized is pelletized, the nucleic acid tagged ferrous compound may be combined with a fertilizer coating before being added to the pelletized fertilizer. The fertilizer coating is preferably an anti-caking agent as described above.

The following number of μg may be combined to create a minima, a maxima, or range for amount of ferrous compound present per 1 kg of fertilizer coating: 0.5 μg, 1 μg, 1.5 μg, 2 μg, 2.5 μg, 3 μg, 3.5 μg, 4 μg, 4.5 μg, 5 μg, 5.5 μg, 6 μg, 7 μg, 8 μg, 9 μg, and 10 μg. For example, the ferrous compound may be present in about 0.1 μg to about 5 μg per kg of fertilizer coating. Preferably, about 3 μg ferrous compound is added to about 1 kg of fertilizer coating.

The nucleic acid taggant may be easily recovered when attached to a ferrous compound by exposure to a magnetic field. The taggant may then be removed from the ferrous compound by placing the nucleic acid tagged ferrous compound in water. The nucleic acid will disassociate from the ferrous compound. The isolated nucleic acid taggant may then be analyzed by conventional methods of DNA analysis.

For example, the DNA taggant is identifiable by any suitable nucleic acid amplification and/or taggant sequence detection technique. Nucleic acid amplification may be accomplished via any technique known in the art, such as, for example, polymerase chain reaction (PCR), loop mediated isothermal amplification, rolling circle amplification, nucleic acid sequence base amplification, ligase chain reaction, or recombinase polymerase amplification. In addition, any known sequence detection and/or identification technique may be used to detect the presence of the nucleic acid taggant such as, for example, hybridization with a taggant-sequence specific nucleic acid probe, an in situ hybridization method (including fluorescence in situ hybridization: FISH), as well as amplification and detection via PCR, such as quantitative (qPCR)/real time PCR (RT-PCR). Isothermal amplification and taggant sequence detection may also be performed with the aid of an in-field detection device such as the T-16 Isothermal Device manufactured by TwistDX, Limited (Hertfordshire, United Kingdom).

### Method of Quantitative Analysis of Nucleic Acid Marked Fertilizer Products

In another embodiment, the present invention provides for a method of quantitative analysis of nuclear acid marked pelletized fertilizer products. This methods includes the steps of: adding a nucleic acid marker to a pelletized fertilizer product during a step in the production process of the fertilizer product, thereby created a nucleic acid marked pelletized fertilizer product; introducing the nucleic acid marked pelletized fertilizer product into the stream of commerce; collecting a known number of pelletized fertilizer product pellets from a single source in the stream of commerce; calculating the sample size necessary to achieve a desired level of statistical certainty; sampling the calculated number of collected fertilizer product pellets to determine the presence or absence of a nucleic acid marker in each pellet; determining whether the collected fertilizer product has been adulterated and/or diluted with counterfeit fertilizer pellets based upon the ratio of the number of pellets that contain a nucleic acid marker to the number of pellets that do not contain a nucleic acid marker.

In one embodiment, the necessary sample size is calculated using the formula n=[1.95̂2]p(1−p)*N/([1.95̂2]p(1−p)+(N−1)Ê2).

Examples have been set forth below for the purpose of illustration and to describe the best mode of the invention at the present time. The scope of the invention is not to be in any way limited by the examples set forth herein.

### EXAMPLES

### Example 1

A nucleic acid taggant was applied to fertilizer pellets via a fertilizer coating. Several kilograms of fertilizer with various concentrations of DNA were made by applying different volumes of nucleic acid concentrate stock solution to a fertilizer coating, which in turn was applied to a pelletized, i.e., granulated fertilizer.
1. 1 kg of NPK (nitrogen, phosphorus, potassium) fertilizer was weighed out using a balance directed into an uncovered bucket having a tightly fitting lid.
2. About 2.4 g of talc was weighed on top of the NPK.
3. The lid was placed on a bucket and the bucket was shaken for about 10 seconds. The samples were mixed in a lidded bucket by shaking by hand in a circular motion to replicate the movement of a coating drum. The bucket was not inverted during mixing.
4. A variable weight of “stock solution” (Lilamin®, pigment, and DNA in quantities listed in Table 1) was added to the bucket by pipette. The solution was dripped across the surface of the granules rather than applied to one location.
5. Novoflow oil (16L205 or 16L207) was removed from a 92° C. oven and between 2-2.5 g was quickly weighed and added to the NPK by disposable pipette drizzled across the surface.
6. The bucket was quickly closed and shaken for 10-15 seconds to disperse the oil and stock solution throughout the NPK. Steps 5 and 6 were performed as quickly as possible to avoid oil cooling below its dropping point.
7. The dispersion was inspected and 100-150 g samples were taken for authentication.
8. The remaining coated fertilizer was tested for stability, dust creation, and compaction to ensure that the methods did not affect the coating efficiency and performance of the NPK.

Stock solutions were made according to Table 1 below.

TABLE 1
Composition of Stock Solution made for Marking

| Stock | Lilamin | Pigment | DNA conc | | | | Viscosity |
|---|---|---|---|---|---|---|---|
| solutions | (g) | (g) | (μL) | Pigment type | % Dye | % DNA | (cps) |
| A | 15.00 | 4.00 | 0 | Clariant Jaune | 21.1% | 0.00% | 620 |
| B | 15.00 | 3.00 | 0 | Clariant Jaune | 16.7% | 0.00% | |
| C | 15.00 | 2.99 | 180 | Clariant Jaune | 16.5% | 0.99% | |
| D | 1.51 | 0.00 | 30 | None | 0.0% | 1.95% | |
| E | 15.00 | 3.00 | 0 | Sico Echt Rot | 16.7% | 0.00% | |
| F | 7.50 | 1.50 | 180 | Clariant Jaune | 16.3% | 1.96% | |
| Lilamin (C) | 10.00 | 5.00 | 0 | Heliogen blue | 33% | 0% | |

Test samples were prepared using the methods describe above and the proportion of materials used for each sample is listed below.

TABLE 2

| | | | | | | Oil | | |
|---|---|---|---|---|---|---|---|---|
| | Fertilizer | | | Stock solution | | | Weight | |
| Test | | Weight | Talc (g) | Ref. | Weight | Type | (g) | Notes |
| 1 | 12 − 12 − 17 + 2 | 1001.60 | 2.40 | — | — | 16L207 | 2.00 | Blank with dye in talc |
| 2 | 12 − 12 − 17 + 2 | 1000.14 | 2.40 | A | 0.90 | 16L207 | 2.00 | Blank 1 (21% yellow dye) |
| 3 | 12 − 12 − 17 + 2 | 1000.00 | 2.39 | B | 1.59 | 16L207 | 2.69 | Blank 2 (17% yellow dye) |
| 4 | 12 − 12 − 17 + 2 | 1000.00 | 2.45 | B | 1.49 | 16L207 | 2.07 | Blank 3 (17% yellow dye)* |
| 5 | 12 − 12 − 17 + 2 | 1000.90 | 2.40 | C | 1.63 | 16L207 | 2.16 | 16 μg DNA concentrate |
| 6 | 12 − 12 − 17 + 2 | 1000.16 | 2.42 | C | 2.01 | 16L207 | 2.49 | 20 μg DNA concentrate |
| 7 | 12 − 12 − 17 + 2 | 1000.00 | 2.44 | C | 2.53 | 16L207 | 2.11 | 25 μg DNA concentrate |
| 8 | 12 − 12 − 17 + 2 | 1000.00 | 2.38 | C | 3.02 | 16L207 | 1.98 | 30 μg DNA concentrate |
| 9 | 12 − 12 − 17 + 2 | 1000.07 | 2.41 | C | 1.01 | 16L207 | 2.11 | 10 μg DNA concentrate |
| 10 | 12 − 12 − 17 + 2 | 1000.02 | 2.42 | C | 0.67 | 16L207 | 2.07 | 7 μg DNA concentrate |
| 11 | 12 − 12 − 17 + 2 | 1000.02 | 2.39 | C | 0.52 | 16L205 | 2.00 | 5 μg DNA concentrate |
| 12 | 12 − 12 − 17 + 2 | 1000.05 | 2.37 | D | 0.50 | 16L207 | 2.10 | 10 μg DNA concentrate, no dye |
| 13 | 12 − 12 − 17 + 2 | 1000.06 | 2.42 | E | 1.61 | 16L207 | 2.14 | Blank (17% red dye) |
| 14 | 16 − 7 − 13 | 999.98 | 2.44 | — | — | 16L207 | 2.65 | Blank 1 with dye in talc |
| 15 | 16 − 7 − 13 | 1000.06 | 2.42 | — | — | 16L207 | 2.22 | Blank 2 with dye in talc* |
| 16 | 16 − 7 − 13 | 999.60 | 2.43 | B | 1.60 | 16L207 | 2.47 | Blank (17% yellow dye) |
| 17 | 16 − 7 − 13 | 1000.03 | 2.43 | F | 0.51 | 16L207 | 2.48 | 10 μg DNA concentrate |
| 18 | 16 − 7 − 13 | 1000.90 | 2.40 | F | 1.04 | 16L207 | 2.42 | 20 μg DNA concentrate |
| 19 | 16 − 7 − 13 | 1000.04 | 2.44 | F | 1.51 | 16L207 | 2.60 | 30 μg DNA concentrate |
| 20 | 16 − 7 − 13 | 1000.00 | 2.44 | F | 2.28 | 16L207 | 2.52 | 45 μg DNA concentrate |
| — | 12 − 12 − 17 + 2 | ~100 | 0.00 | — | 0.00 | — | 0.00 | 12 − 12 − 17 + 2 no treatment |
| — | 16 − 7 − 13 | ~100 | 0.00 | — | 0.00 | — | 0.00 | 16 − 7 − 13 no treatment |
| — | Low porosity | 1000.00 | 2.50 | Lilamin | 0.80 | FL 797 | 2.00 | Sample T5 from test 20 Oct. 2016 |
| | | | | (C) | | | | |

### Conclusions:

Each of the DNA marked pellets were successfully authenticated using various extraction and detection methods including real-time PCR, qPCR and PCR/Capillary Electrophoresis.

### Example 2

In addition to the initial testing of the authentication of marked pellets, several tests were conducted to analyze the transfer of the nucleic acid taggant from the pellets onto unmarked pellets. This was performed by mixing unmarked fertilized pellets in a container with marked pellets from the lab scale trials. The pellets were mixed together at varying ratios (1 kg final mass) and shaken vigorously overnight in a bench top shaker/incubator. After, pellets were placed in bags and left for a week. The bags were shaken again overnight and then randomly sampled. The samples were subjected to various DNA extraction and detection methods.

The marked pellets transferred a small amount of DNA onto the unmarked pellets. There was disparity among marked and unmarked pellets during a series of tests including a 10%, 20%, 30%, 40%, and 50% dilution of the product with unmarked pellets.

### Conclusions:

Using quantitative qPCR, the laboratory successfully determined that there was a discrepancy among marked and unmarked pellets during a series of test including the 10%, 20%, 30%, 40%, and 50% dilution of the marked product with unmarked pellets. The test indicated the ability to detect a dilution of the product in field.

After evaluating the data, it was concluded that the Lilamin-Pigment(dye) and Lilamin-Solagum mixtures provided a stable environment for nucleic acid taggant longevity.

### Example 3

Ferrous beads were used in a 2:1 ratio of wetting solution (comprised of a nucleic acid taggant, antioxidants and wetting agent) to magnetic (ferrous) beads, i.e., 200 mg of beads with 400 μL of wetting solution. The wetting solution specifically contained Sodium Ferulate, EDTA tetrasodium dehydrate, Nonidet P40 wetting agent (NP40) (octylphenoxypolyethoxyethanol), and a nucleic acid taggant.

### Preparation of Solution to Wet Magnaflux Magnaglo Magnetic Beads

### 1. Sodium Ferulate Calculations:

1.1. A final concentration of 0.006 M sodium ferrulate is needed for a 1.00 L batch.
1.2. The mass of sodium ferrulate is determined according to the formula below:
   1.2.1. (216.17 g/mol) (1.00 L) (0.006 M)=1.297 g
      2. EDTA tetrasodium salt dihydrate for Concentrate Calculations:
2.1. A final concentration of 0.003 M is needed for a 1.00 L batch.
2.2. The mass of EDTA tetrasodium salt dihydrate is determined according to the formula below:
   2.2.1. (416.2 g/mol) (1.00 L) (0.003 M)=1.249 g
      3. Nonidet P40 Wetting agent (NP40) Calculations:
3.1. NP40 will be used at 0.01% concentration of the total volume (1.00 L).
3.2. The volume of NP40 is determined according to the formula below:
   3.2.1. (0.01% v/v) (1.00 L)=0.10 mL

A nucleic acid taggant was added to the wetting solution prior to coating the magnetic beads. The DNA concentration may vary.

100 mg, 150 mg, & 200 mg of beads were added to three (03) separate 25 mL scintillation vials and coated with 200 μL, 300 μL and 400 μL of wetting solution, respectively. The samples were allowed to dry overnight in a 40° C. oven to ensure that the samples were completely dry.

DNA Extraction from the Magnetic Beads

The magnetic beads bind the DNA in non-aqueous solutions such as fertilizer coatings. Place the tube containing beads in non-aqueous solution on a large magnet to draw the beads to the bottom. Pipette off the solution leaving the beads behind.
1. Confirmation of DNA binding
   a. Diluted the non-aqueous solution in water and analyze the solution via qPCR for DNA presence. DNA concentration was de minimis, showing successful binding of DNA to magnetic beads in non-aqueous solution.
2. DNA Extraction
   a. Placed the tube containing DNA tagged magnetic beads in water (aqueous solution) on a large magnet to draw the beads to the bottom. Pipetted off the solution leaving the magnetic beads behind.
   b. Diluted the aqueous solution in water and analyze the solution with via qPCR for DNA presence. DNA was detected in the analyzed solution showing successful release of the DNA from the magnetic beads.

1. A method of making a nucleic acid tagged fertilizer, said method comprising:
contacting a nucleic acid taggant with a fertilizer coating to produce a nucleic acid tagged fertilizer coating; and
adding the nucleic acid tagged fertilizer coating to a fertilizer to form a nucleic acid tagged fertilizer,
wherein the fertilizer is pelletized.

2. The method of claim 1, wherein the fertilizer coating is an anti-caking agent.

3. The method of claim 2, wherein the anti-caking agent comprises clay, talc, chalk, petroleum oils, waxes, polyalcohol, fatty amines, cationic surfactants based on fatty amines, alkyl-aryl sulphonates, mineral oils, alkyl phosphoric esters, paraffins, paraffin oil, and combinations thereof.

4. The method of claim 3, wherein the anti-caking agent comprises a fatty amine.

5. The method of claim 1, wherein the adding comprises tumble coating.

6. The method of claim 1, wherein the nucleic acid taggant is present at the level of about 0.1 nanograms to 25 micrograms per kilogram of fertilizer coating.

7. The method of claim 1, wherein the nucleic acid taggant is present at the level of about 5 micrograms per ton of fertilizer.

8. A method of making a nucleic acid tagged fertilizer, comprising:
contacting a nucleic acid taggant, one or more antioxidants, and a wetting agent with a ferrous compound to produce a nucleic acid tagged ferrous compound; and
adding the nucleic acid tagged ferrous compound to a fertilizer coating to form a nucleic acid tagged fertilizer.

9. The method of claim 8, wherein the ferrous compound comprises iron, nickel, cobalt, or alloys thereof.

10. The method of claim 9, wherein the ferrous compound is ferrite.

11. The method of claim 10, wherein the ferrite is coated with a UV fluorescent dye.

12. The method of claim 8, wherein the nucleic acid tagged ferrous compound is present in about 0.1 μg to about 10 μg per ton of fertilizer.

13. The method of claim 8, wherein the one or more antioxidants are selected from the group consisting of sodium ferulate, EDTA, EDTA tetrasodium dehydrate, and combinations thereof.

14. The method of claim 8, wherein the wetting agent is selected from the group consisting of octylphenoxypolyethoxyethanol, sorbitan monolaurate, sorbitan monooleate, polyethylene glycol sorbitan monolaurate, sorbitane trioleate, polyethylene glycol 400, and combinations thereof.

15. The method of claim 8, wherein the ratio of wetting agent to ferrous compound is about 2:1.

16. The method of claim 8, wherein the fertilizer is powdered or pelletized.

17. The method of claim 16, wherein the fertilizer is pelletized and the nucleic acid tagged ferrous compound is combined with a fertilizer coating before being added to the pelletized fertilizer.

18. The method of claim 17, wherein the fertilizer coating is an anti-caking agent selected from the group consisting of clay, talc, chalk, petroleum oils, waxes, polyalcohol, fatty amines, cationic surfactants based on fatty amines, alkyl-aryl sulphonates, mineral oils, alkyl phosphoric esters, paraffins, paraffin oil, and combinations thereof.

19. The method of claim 16, wherein the fertilizer is pelletized and the adding comprises tumble coating.

20. The method of claim 8, wherein the nucleic acid taggant is a DNA taggant.

21. The method of claim 8, wherein the ferrous compound comprises particles of about 1 nanometer to about 500 microns.

22. The method of claim 16, wherein the fertilizer is powdered.

23. A fertilizer composition comprising:
fertilizer and a nucleic acid tagged ferrite wherein the ferrite is present in about 0.1 μg to about 10 μg per ton of fertilizer.

24. The fertilizer composition of claim 23, wherein the nucleic acid tagged ferrite is DNA tagged ferrite.

25. The fertilizer composition of claim 23, wherein the fertilizer is pelletized and coated with an anti-caking agent containing the nucleic acid tagged ferrite.
